(19) United States
(12) Patent Application Publication
(10) Pub. No.: US 2021/0315363 A1
(43) Pub. Date: Oct. 14, 2021

(54) STRAP DISPENSER

(71) Applicant: Briggs and Riley Travelware, LLC, Hauppauge, NY (US)

(72) Inventors: Richard Krulik, Northport, NY (US); Nicholas Byrne, Oakdale, NY (US); Hayley Lawson, Long Beach, NY (US); Ty Bustamante, Austin, TX (US); Spenser Vogel, Englewood, CO (US); Adam Courter, Long Beach, NY (US)

(21) Appl. No.: 17/226,768

(22) Filed: Apr. 9, 2021

Related U.S. Application Data
(60) Provisional application No. 63/008,086, filed on Apr. 10, 2020.
(60) Provisional application No. 63/016,651, filed on Apr. 28, 2020.
(60) Provisional application No. 63/055,108, filed on Jul. 22, 2020.

(51) Int. Cl. A45F 3/04; A61L 2/26; A61L 2/00; A61K 8/02

(52) U.S. Cl. CPC A45F 3/04; A61L 2/26; A45F 2003/003; A61L 2/0094; A61K 8/0208; A61L 2/0088

(57) ABSTRACT

A strap for a backpack, briefcase, or other tote article, including such article, having a sanitizer dispenser is disclosed.

### CROSS REFERENCE TO RELATED APPLICATIONS

This application claims priority benefit under 35 U.S.C. § 119 to the following U.S. provisional applications: U.S. Ser. No. 63/008,086 filed Apr. 10, 2020; U.S. Ser. No. 63/016,651 filed Apr. 28, 2020; and U.S. Ser. No. 63/055,108 filed Jul. 22, 2020, the entire contents of each of which are incorporated herein by reference for any and all purposes.

### FIELD

The disclosure relates to strap for backpacks and briefcases and other tote articles having sanitizer dispensing capability.

### BACKGROUND

Backpacks having hydration features include those with tubes that are connected to beverage reservoirs located within or on the backpack, such as bladders filled with water. The tubes typically extend freely out of the backpack and are configured to dispense water by, for example, having a push-pull cap at the end of the tube, the cap being inserted into the mouth and water sipped though the tube as though drawn through a straw.

Health concerns and cleanliness considerations where convenient access to a sanitizer is desired has given rise to sanitizers being housed in small containers that are clipped onto bags, such as diaper bags.

### SUMMARY

In one aspect the disclosure is directed to a backpack, which is defined herein to include briefcases, that is configured to dispense one or more sanitizers.

### BRIEF DESCRIPTION OF DRAWINGS

FIG. 1 is a front view of a first embodiment of the disclosure showing a pair of backpack straps and components for a sanitizer dispenser.

FIG. 2 is a front view of second embodiment of the disclosure showing a pair of backpack straps and components for a sanitizer dispenser.

FIG. 3 is a front view of a third embodiment of the disclosure showing a pair of backpack straps and components for a sanitizer dispenser.

FIGS. 4A to 4G are a perspective view of a fourth embodiment of the disclosure showing a sanitizer dispenser details (FIGS. 4A to 4E) in conjunction with a backpack (FIGS. 4F, 4G).

FIG. 5 is a front view of a fifth embodiment of the disclosure showing a pair of backpack straps and components for a sanitizer dispenser.

FIG. 6 is a front view of a sixth embodiment of the disclosure showing a pair of backpack straps and components for a sanitizer dispenser.

FIGS. 7A to 7C are plan views of an embodiment of a tube and component parts as used on the practice of FIGS. 5, 6, and 7.

FIGS. 8A to 8C depict a user dispensing sanitizer form an embodiment of the disclosure.

FIGS. 9A to 9E front view of a seventh embodiment of the disclosure showing a pair of backpack straps and components for a sanitizer dispenser.

FIGS. 10A and 10B show an eighth embodiment of the disclosure showing a front view of a backpack strap and a rotating sanitizer dispenser nozzle. FIGS. 10C and 10D show a perspective view of the rotating dispenser nozzle of FIGS. 10A, 10B in an up and down position.

FIGS. 11A to 11C show a ninth embodiment of the disclosure.

FIGS. 12A to 12 C show a tenth embodiment of the disclosure comprising a strap with an enlarged portion.

FIGS. 13A to 13 C show an eleventh embodiment of the disclosure comprising a strap with an enlarged portion.

### DETAILED DESCRIPTION

In one embodiment, the backpack includes one or more tubes each connected at one end to one or more bladders or other containers that contain the sanitizer and are located within the backpack or within the straps themselves. The one or more tubes are disposed partly or entirely within one or more straps of the backpack, e.g. shoulder and/or waist straps. The dispensing end of a respective tube protrudes from the respective strap preferably at a location proximate the hands or otherwise conveniently situated for local application, the dispensing end of the tube comprising a nozzle or applicator suitable to permit adequate flow of the sanitizer. The bladder can be a pre-pressurized to facilitate dispensing, or can be pressurized before or during use by way of a cartridge comprising a pressurized gas, e.g. air, which urges the sanitizer out of the bladder on dispensing, or a pressure bulb attached to the bladder which a user pump to push are into the bladder pressurizing it. Gravity can also be used to dispense the sanitizer from the bladder. The bladder can comprise a flexible or semi-flexible material, polymers and the like, e.g. rubber; or can comprise a hard container, e.g. a metal canister, which can be pre-filled with sanitizer and then pressurized for use. The bladder whether flexible, semi-flexible or hard can be divided into compartments, each having its own sanitizer or conveying liquid. The bladder may be refillable, e.g. refillable with sanitizer or conveying liquid, to permit reuse

The strap or straps may be partly or completely openable either along their width or along their length, e.g. by zipper, Velcro or other means to access the tubes, but need not be. Multiple access zippers and the like may be used. In one practice, the dispensing end of a tube is hidden in the strap, which strap has an opening configured to expose the dispensing end, such opening being a hole or slit sized so that the dispensing end can protrude outwardly from the strap. The opening, e.g. hole, can closed by a button or zipper or other means. In one practice, the hole is opened only for purposes of deploying the dispensing end, e.g. by pulling the dispensing end, or the tube, or a tag attached to the dispensing end or tube such that the dispensing end is revealed sufficient to apply the sanitizer whereafter it is returned to the strap with the hole being then closed. The dispensing end may comprise a spray, a button, e.g. a button valve, that when activated releases the sanitizer, or it can be configured such that pulling on the dispensing end and/or the tube releases the sanitizer. In another embodiment, the strap(s) are hollow and fluid tight and define an interior passageway that is in fluid communication with the bladder, thus eliminating the need for tubes within the straps; in this practice, the strap has the dispensing end incorporated into it at a location proximate the hands.

The sanitizer can be a gel, having a viscosity greater than that of water, or the sanitizer can be a liquid, or a foam. The sanitizer has antimicrobial properties (as used herein “antimicrobial properties” includes antibacterial, antiviral, antifungal, and anti-parasitical properties that decrease the amount of these respective infectious agents). The sanitizer can include alcohol(s), e.g. ethanol, propanol, and/or isopropyl alcohol and the like; and/or other agents having antimicrobial properties such as hydrogen peroxide, povidone-iodine, benzalkonium choride, triclosan and the like, and/or prescription medicaments having antimicrobial properties. Other fluids, such as sun screen are contemplated for use. In one practice, multiple bladders and tubes can be utilized to create a specific sanitizer at the point of dispensing, e.g. the backpack has a bladder containing a fluid with particularly desired antimicrobial properties, and another bladder with a conveying or bulking liquid, e.g. to facilitate hand washing, e.g. glycerol or other such agents or emollients are used, each separately dispensed and admixed at the point of use, e.g. they are separately dispensing into the hands and mixed by rubbing the hands together. In another practice, dispensing can be remote controlled by methods known in the art, e.g. radio frequency controlled valves and the like. This remote control enables dispensing sanitizer to third parties without the need for hand to hand contact. In another aspect, a cell phone app may be used to activate the dispenser. Means for monitoring the amount of sanitizer in the bladder can be employed to advise the user when the bladder is running low.

Exemplary and non-limiting embodiments will now be described by reference to the figures.

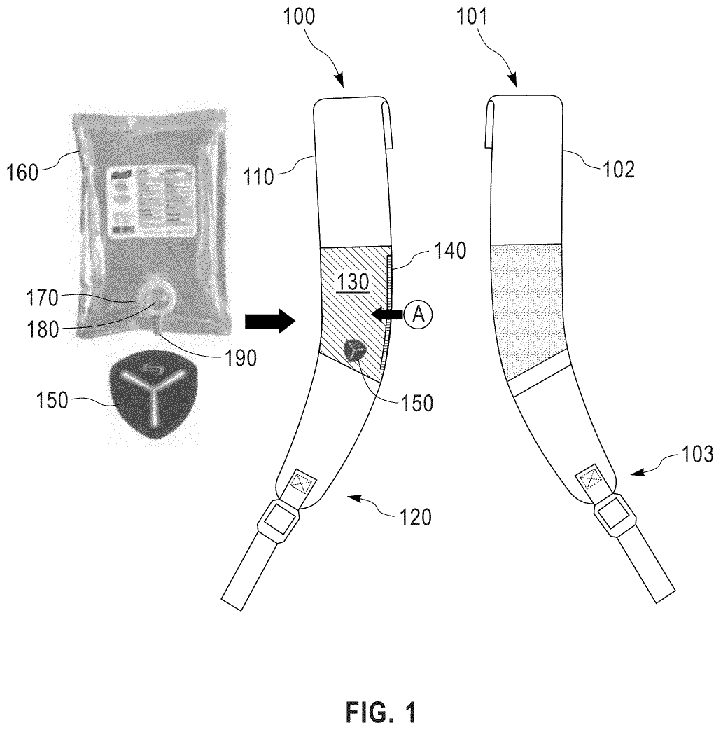

Referring to the FIG. 1, thereat is a pair of backpack straps 101, 101. Strap 100 comprises a top portion 110 attached to the top portion of a backpack (not shown) and bottom portion 120 attached to the bottom portion of a backpack. Strap 101 comprises a top portion 102 attached to the top portion of a backpack (not shown) and bottom portion 103 attached to the bottom portion of a backpack. Strap 100 comprises an internal cavity 130, denoted by hashmarks, proximate the center of the strap, but locatable at any other location on the strap, e.g. proximate the hands, e.g. at or near the bottom portion 120, or e.g. at or near the top portion 110. As shown the strap is of elongated shape and section corresponding to cavity 130 is of substantially rectangular shape, although other geometries of strap and shape of the location of the internal cavity can be employed. Internal cavity 130 is accessible by an openable closure means 140 which can be a zipper, Velcro, buttons or other such means known in the art. As shown, the closure 140 is accessible by arrow A and is on the inside edge of strap 100 but can be on the outside edge of strap 100 or closures can be on both edges of strap 100. Internal cavity 130 is configured to receive bladder 160 which is filled with sanitizer, or alternatively, a package of sanitizer wipes which can be removed, used, and reinserted into cavity 130. In the practice shown, bladder 160 comprises a dispensing end 170, which has a button operated valve 180 as known in the art and a dispensing end 190 from which the sanitizer flows. Bladder 160 is of a material of construction sufficiently squeezable to urge the sanitizer out of the dispensing end 190 when button 180 is pushed and bladder 160 is under pressure, e.g. caused by the user squeezing the bladder or as built up through using the backpack. Internal cavity 130 comprises opening 150, which as shown is a grommet, e.g. comprised of rubber or other deformable or resilient material, but can be other openings as known in the art, e.g. slits and the like, covered or uncovered. Grommet 150 can have a Y-shaped opening as shown, or slits or opening of other shapes and styles. The bladder 160 is placed within internal cavity 130 such that dispensing end 190 and/or button 180 protrudes from opening 16 or is capable of being retrieved from with cavity 130 and accessed from opening 150, e.g. by pulling, when use is desired. In the alternative practice wherein a package of sanitizer wipes is disposed in cavity 130, the wipes can be extruded through grommet 150 so that the package need not be removed. In another practice (not shown), a flap can be attached to strap 100 to cover grommet 150. The flap can be openable by Velcro, snap, zipper of other means. Multiple internal cavities containing the same or different sanitizers or sanitizer components can be employed in strap 100 and the same configuration(s) as herein described can be applied to strap 101 or other straps, separately from strap 100 or in conjunction with strap 100.

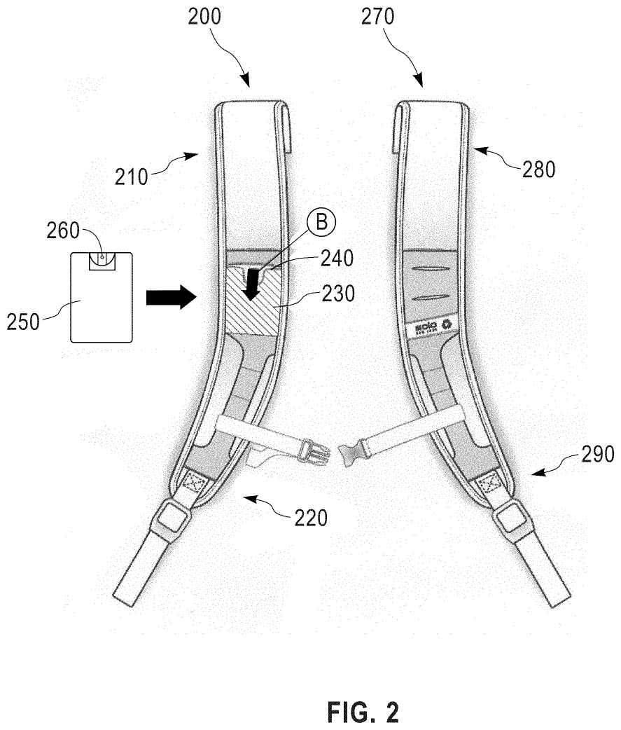

FIG. 2 depicts a pair of backpack straps 200, 270. Strap 200 comprises a top portion 210 attached to the top portion of a backpack (not shown) and bottom portion 220 attached to the bottom portion of a backpack. Strap 270 comprises a top portion 280 attached to the top portion of a backpack (not shown) and bottom portion 290 attached to the bottom portion of a backpack. Strap 200 comprises cavity 230, denoted by hashmarks, proximate the center of the strap, but locatable at any other location on the strap, e.g. proximate the hands, e.g. at or near the bottom portion 220, or e.g. at or near the top portion 210. As shown the strap is of elongated shape and section corresponding to cavity 230 is of substantially rectangular shape, although other geometries of strap and shape of the location of the internal cavity can be employed. As shown, cavity 230 is in the form of a pocket located on the surface of the strap and is accessible from top pocket cutout opening 240, which opening 240 may optionally have closure means e.g. a flap, zipper and the like. Pocket cavity 230 may be a cut out into the actual strap 200 or can be a piece of material overlaid on the strap. Pocket cavity 230 can comprised an elastic material of construction, e.g. rubber such as neoprene, and is configured to receive sanitizer container 250, which is inserted into pocket 230 via arrow B; as depicted, container 250 is a flat refillable spray bottle containing sanitizer as described herein. Other containers may be employed, e.g. pour bottles and the like. Multiple pocket cavities containing the same or different sanitizers or sanitizer components can be employed in strap 200 and the same configuration(s) as herein described can be applied to strap 270 or other straps, separately from strap 200 or in conjunction with strap 200.

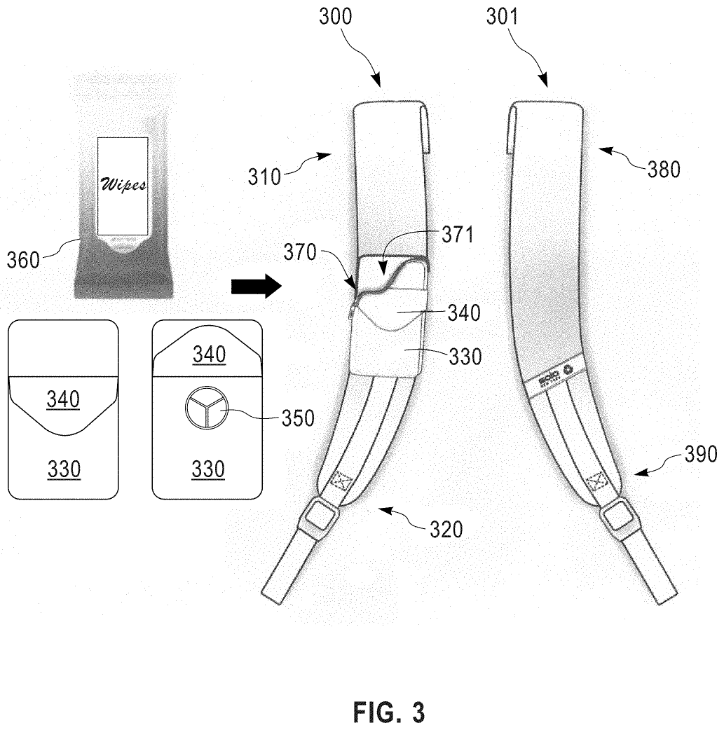

FIG. 3 depicts a pair of backpack straps 300 and 301. Strap 300 comprises a top portion 310 attached to the top portion of a backpack (not shown) and bottom portion 320 attached to the bottom portion of a backpack. Strap 301 comprises a top portion 380 attached to the top portion of a backpack (not shown) and bottom portion 390 attached to the bottom portion of a backpack. Strap 300 comprises a pouch 330 that defines a cavity within the pouch, which pouch 330 as depicted has flap 340 which when in the closed position covers opening 371. Opening 371 is accessible by opening means 370, e.g. a zipper which provides access to the interior of pouch 330. In one practice the zipper at 370 is located across approximately the top half of pouch 330. On the surface of pouch 330 is opening 350 shown in FIG. 3 as a grommet 350 with a Y-shaped slit, although other openings may be employed. Grommet 350 as shown is covered by flap 340. In one practice, a package of sanitizer wipes 360 is inserted into pouch 330 through opening 371 and configured such that a wipe protrudes through grommet 350 and such that removal of one wipe causes the next wipe in package 360 to protrude from grommet 350. Multiple pouches containing the same or different sanitizer wipes can be employed in strap 300 and the same configuration(s) as herein described can be applied to strap 301 or other straps, separately from strap 300 or in conjunction with strap 300.

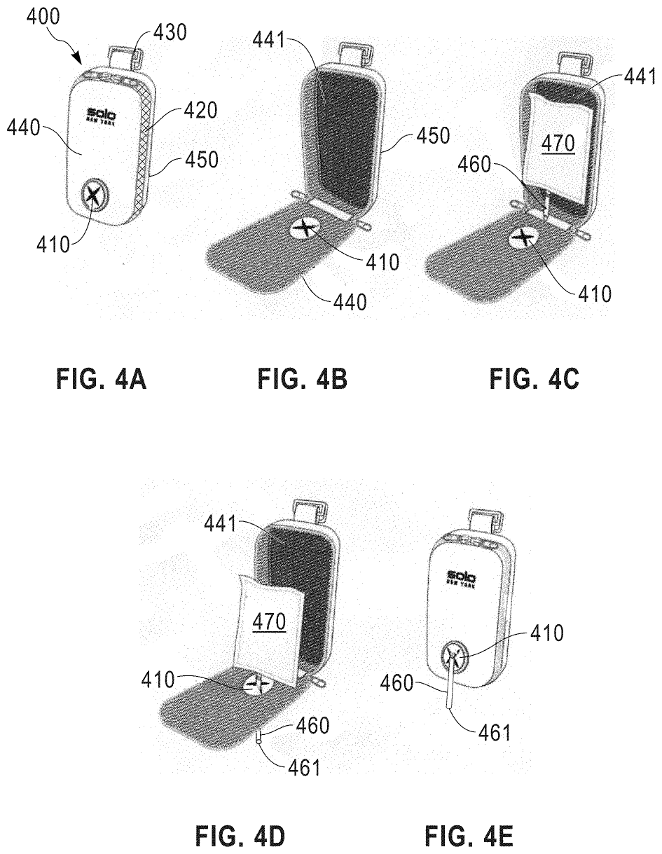

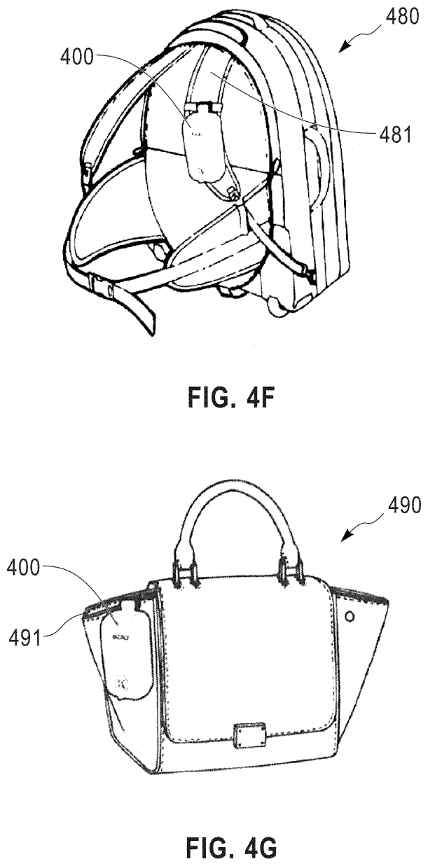

FIGS. 4A to 4E depict an embodiment of a sanitizer container 400 comprising a front portion 440 and a back portion 450 which define internal cavity 441 and are hingedly or otherwise connected together and are openable (FIG. 4B) by opening means, e.g. zipper 420, or snaps or latches and other means known in the art. Container 400 has attachment means 430 which can be clips, loops and the like for ultimate attachment to a backpack, and has opening 410, e.g. on front portion 440, opening 410 can be in the form of a grommet or other design. In practice, a bladder 470 containing a sanitizer is placed within internal cavity 441. Bladder 470 comprises one or more tubes 460, here in the shape of a straw, that is in fluid communication with the sanitizer contents of the bladder and has a dispensing end 461 (FIGS. 4C, 4D). Tube 460 and dispensing end 461 are inserted into an through the grommet 410 so that at least the dispensing end and optionally a portion or tube 460 is protrudes or is accessible from grommet 410 (FIG. 4E) and the front portion 440 and back portion 450 are closed together via zipper 420. Container 400 can then be attached to backpack 480 (FIG. 4F) or to a tote (FIG. 4G) or other bag by loops 430 or can be attached via clips, Velcro or other means known in the art.

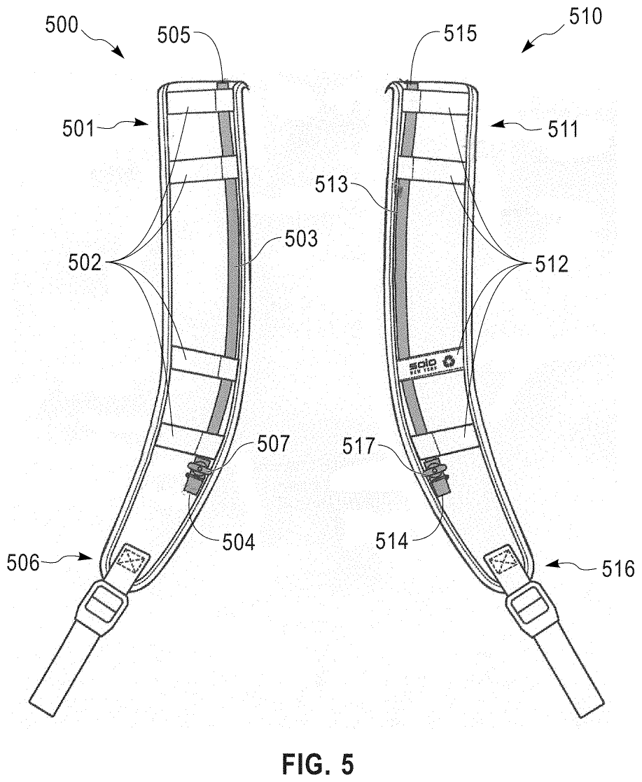

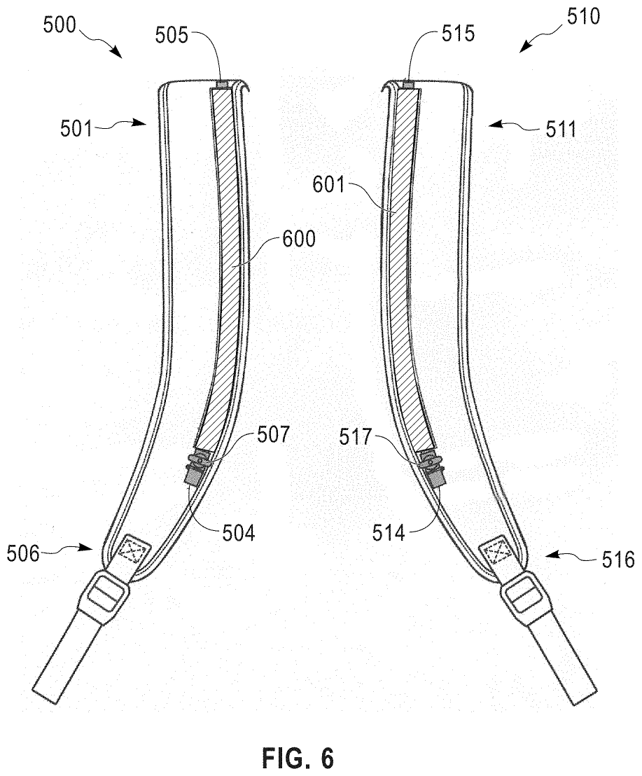

FIGS. 5 and 6 depict a pair of backpack straps 500 and 510 having respective top portions 501 and 511 and respective bottom portions 506 and 516. FIG. 5 depicts a tube 503 and 513 on each of straps 500 and 510 although only one tube or multiple tubes on each strap or on one strap or the other may be employed, the tubes permitting fluid flow therefrom by gravity or by manipulating the tube. As shown in FIG. 5, one or more tube straps 502 and 512 are respectively placed across the straps 500 and 510. These may be of elastic or non-elastic material and are sized to secure tubes 503 and 513 to the face of the strap thereby minimizing or eliminating movement of tubes 503 and 513, but not so tight so as to impede flow of the sanitizer out of the tubes. As shown in FIG. 6, sleeves 600 and 601 are respectively present on straps 500 and 510, the sleeves being substantially equal to or less than the length of the tubes 503 and 513, or segmented. The sleeves 600, 601 may be attached separately to the strap or integral therewith, and can be made of fabric, or polymeric material.

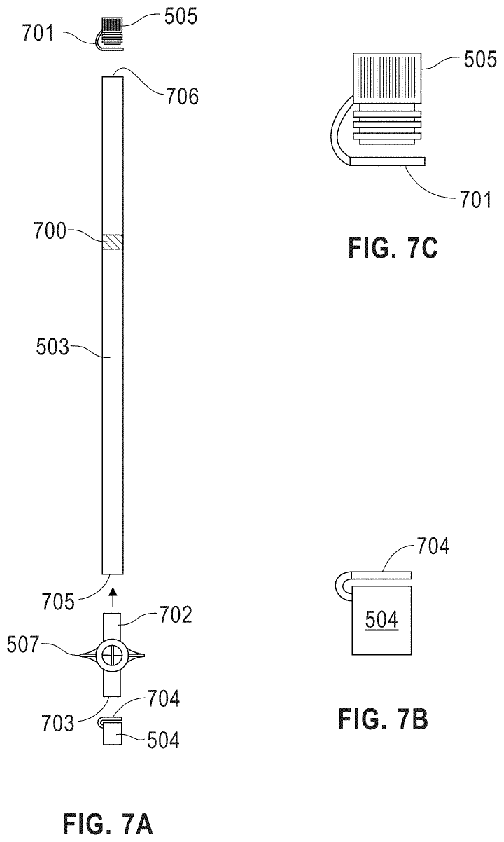

Tubes 503 and 513 are filled or partly filled with sanitizer or other fluid and can be of a material of construction that is opaque or preferably sufficiently transparent, including clear or tinted, so as to see the level of how much sanitizer is in the tube. In the practice shown, tubes 503, 513 are polymeric, and preferably FDA approved materials, such as polyvinyl chloride (PVC) and the like, and are preferably flexible along their length so as to conform to the backpack straps 500 and 510 and movements created by the user. In another aspect, tubes 503 and 513 can be soft enough so as to squeeze or pinch the sidewalls together in order to urge sanitizer or other fluid out of the tube, e.g. if the sanitizer or other fluid is sufficiently viscous such that it does not easily flow by gravity. Tubes 503 and 513 have valves 507 and 517 respectively at the discharge end which can be covered by removable caps 504 and 514 respectively. Valves 507 and 517 can be of designs known in the art, e.g. quarter turn valves and the like. As shown in FIGS. 7A, 7B, 7C, the valve, shown as 507 for convenience, has valve inlet portion 702 which is configured to sealingly fit into tube outlet 705, e.g. by friction fit or other means. Valve outlet portion 703 provides the sanitizer discharge end for the user. Removable discharge end cap 504 is configured to cover the discharge end 703 when not in use, and can be connected to the valve outlet portion 703 by tether 704 which has a ring connection with opening therethrough sized to fit portion 703; other connection means as known are serviceable. In one practice, tube 503 is filled with sanitizer through tube inlet hole 706 and can be fully filled or filled to a particular level that coincides with mark 700 that is on the tube. Removable inlet cap 505 is sized to sealingly fit into inlet hole 706, e.g. by friction fit or other means, and can be attached to tube 503 by tether 701 which has a ring connection with opening therethrough sized to tube 503; other connection means as known are serviceable.

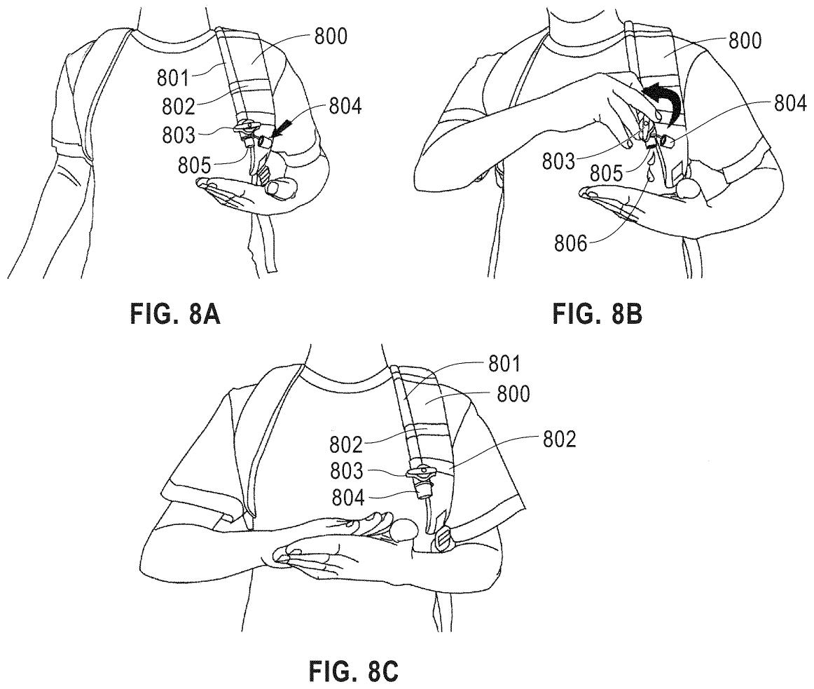

FIGS. 8A, 8B, 8C depict use of an embodiment whereby tube 801 contains e.g. sanitizer and is secured to strap 800 by one or more tube straps 802. In FIG. 8A, discharge end cap 804 is removed by the user thus exposing discharge outlet 805 of tube 801, the user placing a hand under outlet 805. In FIG. 8B, the user opens valve 803 using the other hand whereafter sanitizer 806 flows by gravity into the hand placed beneath discharge outlet 805. In FIG. 8C, the valve 803 has been closed and end cap 804 replaced, the user applying the sanitizer to both hands.

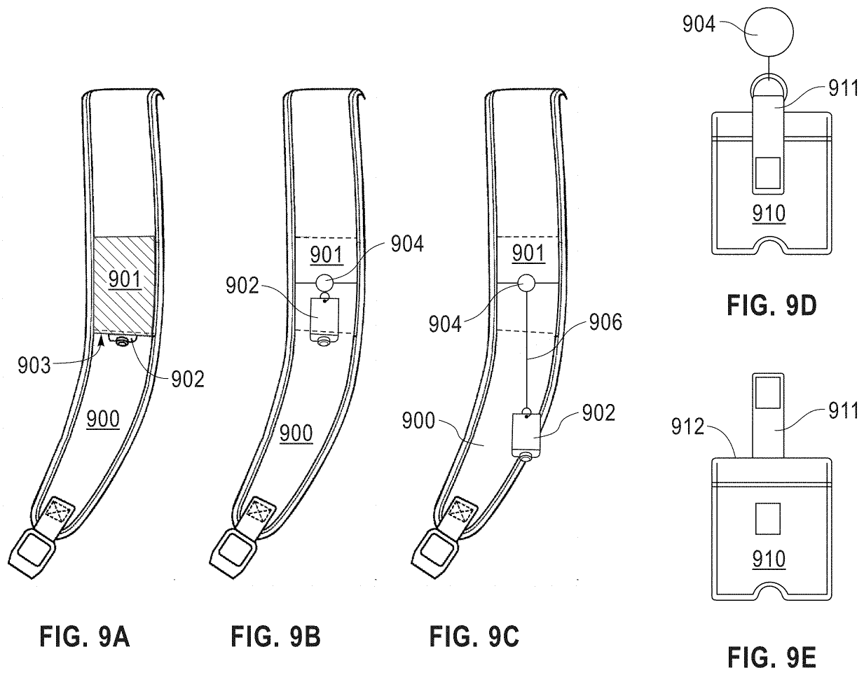

FIGS. 9A, 9B, 9C, and 9D depict another embodiment whereby a backpack strap 900 comprises an internal cavity 901 formed by a pocket internal to the strap or pouch formed by an overlay of material, e.g. fabric or attached by Velcro and the like, as depicted in FIG. 9A by hash marks. The internal cavity 901 is accessible by opening 903. Within the internal cavity 901 is container 902 (which can be refillable) of sanitizer or other fluid and which is connected to a retractable line 906, e.g. string or wire and the like, which line 906 is attached to a key clip 904 which in turn is attached to strap 900 under fabric covering the internal cavity 901; the key clip 904 can be attached to a seam or piece of webbing within the internal cavity or on the covering fabric. FIGS. 9B and 9C show the strap with the fabric covering removed to reveal the internal cavity 901 wherein the key clip 904 is attached to the strap 900, with FIG. 9B showing container 902 in retracted position and FIG. 9C showing it in extended position. In one practice, only the tip of container 902 extends past opening 903, a user grabs the tip and pulls the container out of internal cavity 901 which extends line 906; after use, line 906 retracts container 902 back into internal cavity 901. FIGS. 9D and 9E show an embodiment whereby a container case 910 is attached to the key clip 904 (line 906 in retracted position). Container case 910 can hold a sanitizer container which is inserted into opening 912 which can be closed or covered by flap 911 using Velcro and the like. In use, the container case 910 is pulled out of internal cavity 901 extending line 906 and the user removes the sanitizer container (not shown) therefrom and replaces it after use whereafter it retracts back into cavity 901.

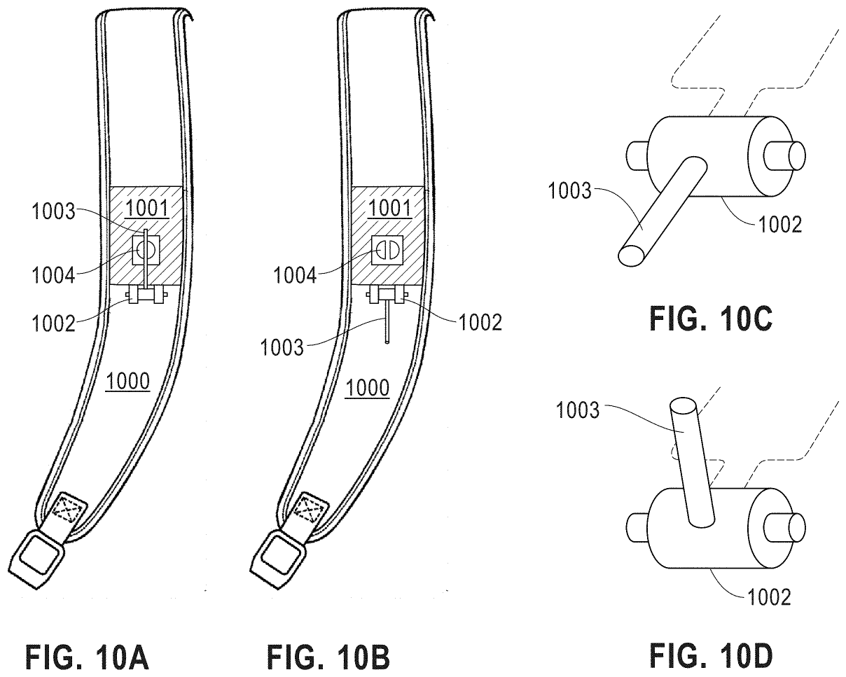

FIGS. 10A and 10B show backpack strap 1000 having internal cavity 1001 indicated by hashmarks within which is a sanitizer dispenser container or bladder (not shown; as herein elsewhere described) having an outlet attached to a rotating dispenser nozzle 1002 having an elongated dispensing stem 1003 wherefrom the sanitizer issues. In FIG. 10a the elongated dispensing stem is in a closed position by being clipped into clip holder 1004 comprised of e.g. two opposing abutments the space between which the dispensing stem 1003 securely fits. FIG. 10B shown the dispensing stem 1003 rotated out of clip 1004 and ready for use as herein described. FIGS. 10C and 10D respectively show dispensing stem 1003 in a ready for use position and in an opposed rotated position (clip 1004 not shown), and connected to and in fluid communication with the sanitizer dispensing container or bladder shown in broken lines.

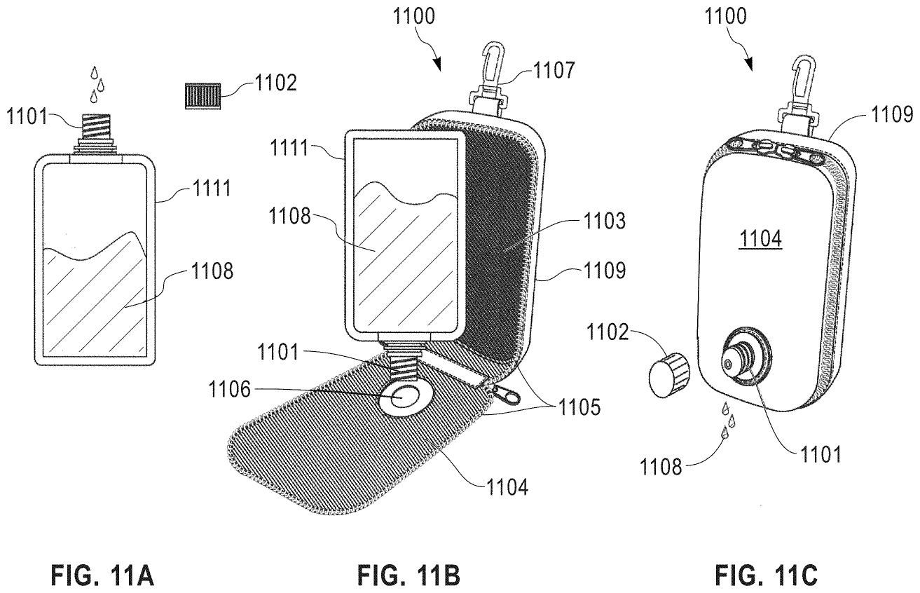

FIGS. 11A to 11C depict an embodiment of a sanitizer container 1100 comprising a front portion 1104 and a back portion 1109 which define internal cavity 1103 and are hingedly or otherwise connected together and are openable (FIG. 11B) by opening means, e.g. zipper 1105, or snaps or latches and other means known in the art. Container 1100 has attachment means, shown as clip 1107 which can also be loops and the like for ultimate attachment to a backpack, and has opening 1106, e.g. on front portion 1104, opening 410 is formed to permit the threaded dispenser outlet 1101 to protrude externally therefrom. Sanitizer bladder 1111 containing sanitizer 1108 is sized to fit within interior cavity 1003 and can be of rectangular or other shape. In practice, bladder 1111 containing a sanitizer 1108 is placed within internal cavity 1103 and the threaded dispenser outlet is placed through opening 1106. The threaded dispenser outlet 1101 can be located on the side of bladder 1111 so that it aligns with opening 1006 when placed in cavity 1103, or the bladder can be sufficiently flexible such that the threaded outlet 1101 can be manipulated through opening 1106 within impeding outward flow of the sanitizer. Once the bladder is situated in cavity 1103 and threaded outlet 1101 is secured through opening 1106, the front and back portions 1104 and 1109 can be closed and removable cap 1102 placed over the threaded outlet. Container 1100 can then be attached to backpack or to a tote or other bag by clip 1107, or Velcro or other means known in the art. The outer surface of back portion 1109 may also have a clip or other means of attachment to the backpack or strap.

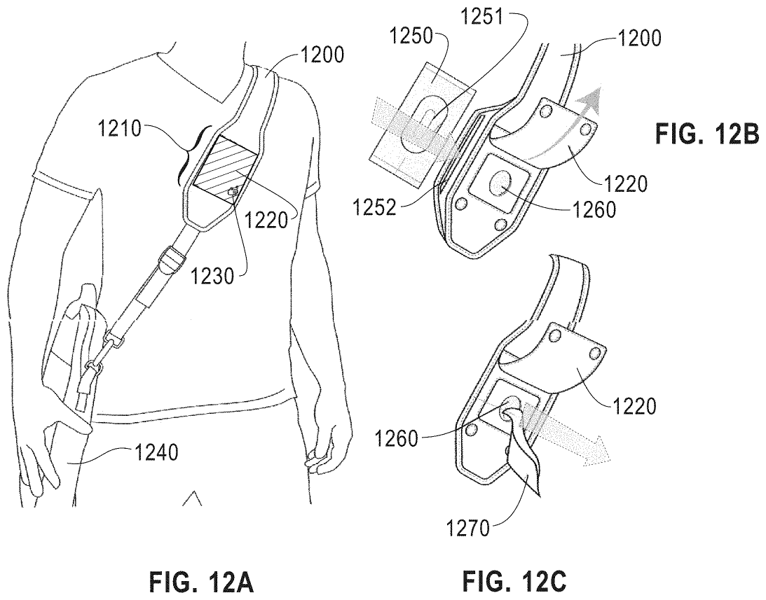

FIGS. 12A to 12C depict an embodiment of a strap dispenser comprising a strap 1200 having an enlarged portion 1210 which comprises an internal cavity (shown in hashmarks) that is accessible from the edge of the strap via edge opening 1252 which is optionally comprises a closure such as a zipper or other closure means (not shown). In this embodiment, strap 1200 is attached to briefcase 1240 although backpacks and other tote articles are contemplated. Enlarged portion 1210 further comprises dispensing outlet 1260 covered by optional flap 1220. Sanitizer dispenser 1250 shown as a wipe dispenser, having dispenser outlet 1251, is disposed into the internal cavity via edge opening 1252 so that wipe dispenser outlet 1251 is in communication with dispensing outlet 1260 of the enlarged portion.

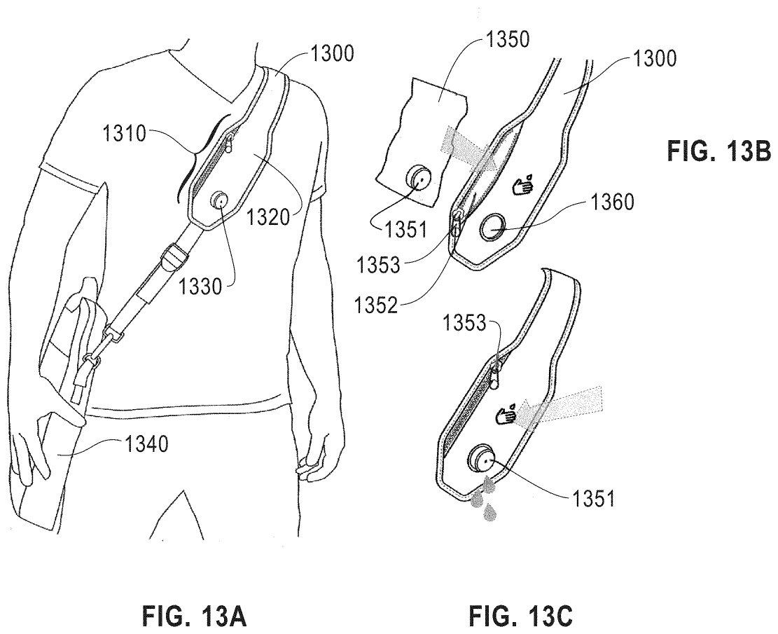

FIGS. 13A to 13C depict another practice wherein strap 1300 has enlarged portion 1310 which comprises an internal cavity accessible from the edge of strap 1300 via edge opening 1352 which optionally comprises zipper 1353 or other closure means. In the embodiment shown, strap 1300 is attached to briefcase 1340 although backpacks and other tote articles are contemplated. Enlarged portion 1310 comprises dispensing outlet 1360, such as a hole through the outer surface to enlarged portion 1301 to the internal cavity. Sanitizer dispenser 1350, shown as a bladder containing e.g. a liquid or gel or foam sanitizer and having dispenser outlet 1351, is disposed into the internal cavity so dispenser outlet 1351 protrudes from outlet 1360 of the enlarged portion. Operationally, the user presses on the enlarged portion (shown as a hand in FIGS. 13B and 13C) to compress the bladder thereby urging the sanitizer to flow from protruding dispenser outlet 1351.

What is claimed is:

1. A sanitizer dispenser strap for a backpack comprising:
a backpack body comprising a top backpack portion, and a bottom backpack portion;
a shoulder strap having a top portion attached to the top backpack portion, and a bottom portion attached to the bottom backpack portion, the shoulder strap comprising an internal cavity accessible from an opening on the outer surface of the shoulder strap, and an outlet in communication with the internal cavity; and
a sanitizer dispenser comprising a sanitizer, the sanitizer dispenser disposed within the internal cavity, the sanitizer dispenser having a dispensing end located at the outlet, the dispensing end configured to dispense the sanitizer from the shoulder strap.

2. The strap of claim 1 wherein the sanitizer is a liquid, a gel, or a foam; and the dispenser is tube containing said sanitizer.

3. The strap of claim 2 wherein the dispensing end of the tube protrudes from the outlet.

4. The strap of claim 3 wherein the tube comprises a valve at the dispensing end.

5. The strap of claim 3 wherein the end of the tube distal from the dispensing end comprises a removable cap.

6. The strap of claim 3 wherein the end of the tube distal from the dispensing end is attached to and in fluid communication with a bladder that contains the sanitizer, the bladder disposed within the internal cavity.

7. The strap of claim 6 wherein the bladder comprises a material of construction sufficiently squeezable to urge to the sanitizer to flow through the tube and out the dispensing end when the bladder is compressed.

8. The strap of claim 1 wherein the sanitizer comprises a sanitizer wipe.

9. The strap of claim 8 wherein the sanitizer wipe protrudes through the outlet.

10. The strap of claim 8 wherein the sanitizer dispenser comprises a package of sanitizer wipes.

11. The strap of claim 1 wherein the sanitizer dispenser is a spray bottle or a pour bottle and is removably disposed with the internal cavity.

12. A sanitizer dispenser strap for a backpack comprising:
a backpack body comprising a top backpack portion, and a bottom backpack portion;
a shoulder strap having a top portion attached to the top backpack portion, and a bottom portion attached to the bottom backpack portion, the shoulder strap comprising (i) a sleeve attached to the shoulder strap, the sleeve situated axially to the top backpack portion and the bottom backpack portion, or (ii) one or more tube straps attached to the shoulder strap, the one or more tube straps situated transversely to the top backpack portion and the bottom backpack portion; and
a dispensing tube containing a sanitizer, the tube having a dispensing end and a capped end distal from the dispensing end, the dispensing tube (i) disposed within the sleeve, or (ii) disposed under the one or more tube straps.

13. The strap of claim 12 wherein the sanitizer is a liquid, a gel, or a foam.

14. The strap of claim 12 wherein the dispensing end of the tube protrudes from (i) the sleeve or (ii) the tube strap most proximate to the bottom portion of the shoulder strap.

15. The strap of claim 12 wherein the dispensing tube comprises a valve at the dispensing end.

16. The strap of claim 12 wherein the dispensing tube comprises a removable cap at the dispensing end.

17. A sanitizer dispenser strap for a backpack comprising:
a backpack body comprising a top backpack portion, and a bottom backpack portion;
a shoulder strap having a top portion attached to the top backpack portion, and a bottom portion attached to the bottom backpack portion, the shoulder strap comprising a pouch located on the outer surface of the shoulder strap, the pouch defining an internal pouch cavity that is accessible by an opening on an edge of the pouch, the pouch having a pouch outlet in communication with the pouch cavity; and
a sanitizer dispenser comprising sanitizer wipes, the sanitizer dispenser disposed within the pouch cavity, the sanitizer wipes accessible from the pouch outlet.

18. The strap of claim 17 wherein the pouch comprises a flap covering the pouch outlet.

19. A sanitizer dispenser strap for a backpack comprising:
a backpack body comprising a top backpack portion, and a bottom backpack portion;
a shoulder strap having a top portion attached to the top backpack portion, and a bottom portion attached to the bottom backpack portion, the shoulder strap comprising an internal cavity accessible from an opening on the outer surface of the shoulder strap; and
a sanitizer dispenser comprising a sanitizer, the sanitizer dispenser having a first position that is disposed within the internal cavity, and a second position that is extended outward from the opening.

20. The strap of claim 19 wherein the sanitizer dispenser is movable from the first position to the second position by a retractable line.

21. The strap of claim 20 wherein the retractable line is attached to a key clip secured to the strap within the internal cavity.

22. A sanitizer dispenser strap for a backpack comprising:
a backpack body;
a shoulder strap attached to the backpack body; and
a sanitizer container comprising an opening, an internal cavity, a bladder containing sanitizer disposed within the internal cavity, a tube in fluid communication with the bladder, the tube having a dispensing end in fluid communication with the opening, the sanitizer container attached to the shoulder strap.

23. The strap of claim 22 wherein the sanitizer container is removably attached to the shoulder strap.

24. The strap of claim 23 wherein the sanitizer container is removably attached to the shoulder strap by a clip or a loop through which the shoulder strap is disposed.

25. The strap of claim 22 wherein the backpack body comprises a briefcase.

26. The strap of claim 22 wherein the backpack body comprises a top backpack portion, and a bottom backpack portion; and the shoulder strap has a top portion attached to the top backpack portion, and a bottom portion attached to the bottom backpack portion.

27. A sanitizer dispenser strap for a backpack comprising:
a backpack body;
a shoulder strap attached to the backpack body, the shoulder strap having an enlarged portion within which is an internal cavity accessible by an opening on the side of the enlarged portion, the enlarged portion having an outlet in fluid communication with the internal cavity; and
a sanitizer dispenser comprising a sanitizer, the sanitizer dispenser disposed within the internal cavity, the sanitizer dispenser having a dispensing end located at the outlet, the dispensing end configured to dispense the sanitizer from the shoulder strap.

28. The strap of claim 27 wherein the backpack body comprises a briefcase.

29. The strap of claim 27 wherein the backpack body comprises a top backpack portion, and a bottom backpack portion; and the shoulder strap has a top portion attached to the top backpack portion, and a bottom portion attached to the bottom backpack portion.

30. The strap of claim 27 wherein the sanitizer is a liquid, a gel, or a foam; and the dispenser is tube containing said sanitizer.

31. The strap of claim 27 wherein the sanitizer comprises a sanitizer wipe.

32. The strap of claim 27 wherein the outlet is covered by a flap.
